(19) United States
(12) Patent Application Publication
(10) Pub. No.: US 2021/0394776 A1
(43) Pub. Date: Dec. 23, 2021

(54) NOTIFICATION CONTROL DEVICE, NOTIFICATION DEVICE, NOTIFICATION SYSTEM, AND NOTIFICATION CONTROL METHOD

(71) Applicant: Mitsubishi Electric Corporation, Tokyo (JP)

(72) Inventors: Shogo OKAMOTO, Tokyo (JP); Gentaro WASHIO, Tokyo (JP); Kimika IKEGAMI, Tokyo (JP); Takumi SATO, Tokyo (JP); Tetsuro NISHIOKA, Tokyo (JP); Mizuki HIGUCHI, Tokyo (JP); Masanobu OSAWA, Tokyo (JP)

(73) Assignee: Mitsubishi Electric Corporation, Tokyo (JP)

(21) Appl. No.: 17/279,139

(22) PCT Filed: Nov. 19, 2018
(86) PCT No.: PCT/JP2018/042681

§ 371 Date: Mar. 24, 2021

(51) Int. Cl. B60W 50/14; B60W 30/095; B60W 40/09

(52) U.S. Cl. CPC B60W 50/14; B60W 30/095; B60W 2554/4049; B60W 2050/143; B60W 40/09

(57) ABSTRACT

An information acquiring unit acquires information from a detection device mounted on a vehicle. A near accident determining unit determines whether or not a near accident has occurred at the vehicle using the information acquired by the information acquiring unit. A near accident cause classifying unit classifies the cause of the near accident determined by the near accident determining unit. A near accident level determining unit determines the level of the near accident determined by the near accident determining unit and also determines whether or not the level of the near accident exceeds a corresponding one of predetermined threshold levels. A notification destination determining unit determines a target individual to be notified of information regarding the near accident that is determined to exceed the corresponding threshold level by the near accident level determining unit. In this configuration, the threshold levels are set for respective combinations each of which is a combination of a cause and a target individual.

### TECHNICAL FIELD

The present invention relates to a notification control device, a notification device, a notification system, and a notification control method for notifying a near accident that has occurred at a vehicle.

### BACKGROUND ART

Patent Literature 1 describes a cause analysis device that analyzes the cause of an accident or a near accident that has occurred at a vehicle and appropriately issues a warning or the like to a driver. The cause analysis device first compares vehicle information transmitted from the vehicle with vehicle information accumulated for accidents and near accidents that have occurred in the past and thereby identifies commonalities. Subsequently, the cause analysis device estimates whether or not an accident or a near accident that has occurred at the vehicle is attributable to the environment and whether or not the accident or the near accident is attributable to the driver on the basis of the identified commonalities. Then, the cause analysis device determines the main cause of the accident or the near accident of the vehicle on the basis of the estimation result.

### CITATION LIST

### Patent Literature

Patent Literature 1: JP 2016-71492 A

### SUMMARY OF INVENTION

### Technical Problem

The cause analysis device described in Patent Literature 1 analyzes the causes of an accident or a near accident that has occurred at a vehicle and issues a warning to the driver. However, there is a disadvantage that notification cannot be performed with appropriate distinction among target individuals, for example, in a case where a target to be warned is a driver or an individual other than the driver or is a driver who has different driving tendencies.

Cases where it is necessary to perform notification with distinction among target individuals include, for example, a case where the target to be notified is the driver or an individual other than the driver. For example, let us assume that a manager of a transportation company manages multiple drivers. A driver needs to be immediately warned by a notification device installed in the vehicle and strive for safe driving, when a near accident occurs. When a driver causes a near accident, the manager immediately receives a notification of the near accident from the vehicle of the driver, checks the state of the near accident, and gives direct guidance to the driver by telephone or other means. However, the number of notifications is very large if the manager receives notifications of all near accidents, and thus it is laborious to check and to give guidance for all of them. Thus, it is necessary to limit near accidents to be notified to only those that are important to some extent. However, the cause analysis device described in Patent Literature 1 cannot perform notification with distinction between a driver and a manager.

Furthermore, in a case where targets to be notified of near accidents are drivers, for example, driving tendencies are different for each driver such as drivers who frequently doze while driving and drivers who do not doze while driving. In this case, it is necessary to perform notification for the drivers who frequently doze while driving with distinction from drivers who do not doze while driving, by setting a stricter determination degree on a doze sensor that is used for warning of near accidents. However, the cause analysis device described in Patent Literature 1 cannot perform notification with distinction between drivers who have different driving tendencies.

The present invention has been made in order to solve the above-mentioned disadvantage, and an object of the present invention is to provide appropriate notification with distinction among target individuals.

### Solution to Problem

A notification control device according to the present invention includes: an information acquiring unit for acquiring information from a detection device mounted on a vehicle; a near accident determining unit for determining whether or not a near accident has occurred at the vehicle using the information acquired by the information acquiring unit; a near accident cause classifying unit for classifying a cause of the near accident determined by the near accident determining unit into a corresponding one of causes; a near accident level determining unit for determining a level of the near accident determined by the near accident determining unit and determining whether or not the level of the near accident exceeds a corresponding one of predetermined threshold levels; and a notification destination determining unit for determining one of target individuals who is to be notified of information regarding the near accident that is determined to exceed the corresponding one of the predetermined threshold levels by the near accident level determining unit, in which the predetermined threshold levels are set for respective combinations each of which is a combination of a corresponding one of the causes and a corresponding one of the target individuals.

### Advantageous Effects of Invention

According to the present invention, it is possible to perform appropriate notification with distinction among target individuals since notification is made to a target individual when the level of a near accident that has occurred at a vehicle exceeds a corresponding one of threshold levels set for respective combinations each of which is a combination of a corresponding one of causes of near accidents and a corresponding one of the target individuals to be notified.

### BRIEF DESCRIPTION OF DRAWINGS

FIG. 1 is a block diagram illustrating an exemplary configuration of a notification device according to a first embodiment.

FIG. 2 is a diagram illustrating an exemplary hardware configuration of the notification device according to the first embodiment.

FIG. 3 is a block diagram illustrating an exemplary configuration of a notification system according to the first embodiment.

FIG. 4A is a diagram illustrating an example of near accident information displayed by a notification unit in the first embodiment.

FIG. 4B is a diagram illustrating an example of near accident information displayed by the notification unit in the first embodiment.

FIG. 4C is a diagram illustrating another example of near accident information displayed by the notification unit in the first embodiment.

FIG. 4D is a diagram illustrating a display example of details of near accident information selected on the exemplary screen of FIG. 4C.

FIG. 5 is a flowchart illustrating an operation example of the notification system according to the first embodiment.

### DESCRIPTION OF EMBODIMENTS

To describe the invention further in detail, an embodiment for carrying out the invention will be described below with reference to the accompanying drawings.

### First Embodiment

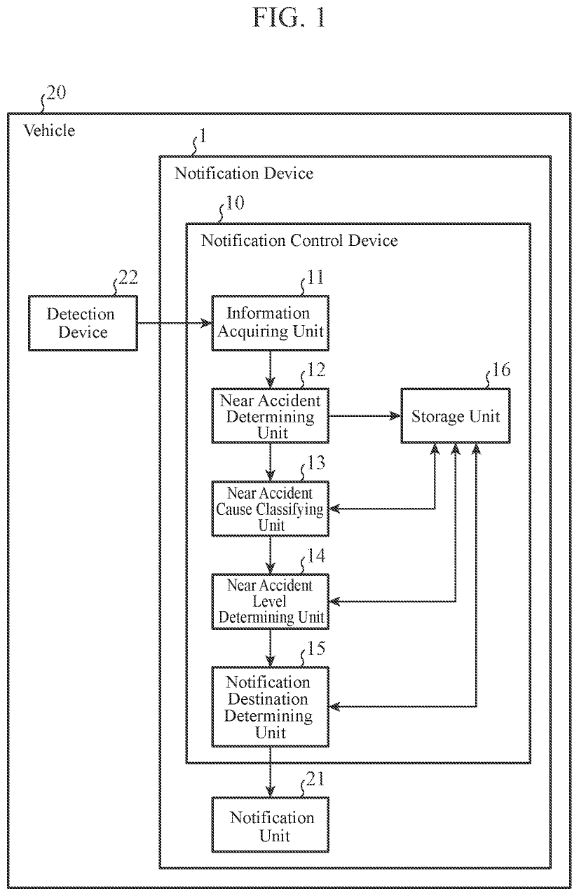

FIG. 1 is a block diagram illustrating an exemplary configuration of a notification device 1 according to a first embodiment. The notification device 1 and a detection device 22 are mounted on a vehicle 20. The notification device 1 includes a notification control device 10 and a notification unit 21. The notification control device 10 includes an information acquiring unit 11, a near accident determining unit 12, a near accident cause classifying unit 13, a near accident level determining unit 14, a notification destination determining unit 15, and a storage unit 16.

The detection device 22 detects information on the driver and the surroundings of the vehicle that is necessary for determining whether or not a near accident has occurred at the vehicle 20. The detection device 22 includes, for example, a driver monitoring device that detects the orientation of the face, a line of sight, the eye-opening degree, and the like of the driver, a camera that captures an image of the driver's face, a camera that captures the surroundings of the vehicle 20, a sensor that detects vehicles, obstacles, and the like around the vehicle 20, an in-vehicle device such as a navigation device that acquires position information of the vehicle 20, road information, map information, etc., and an in-vehicle device that acquires controller area network (CAN) information such as the speed and the steering angle of the vehicle 20. Furthermore, the detection device 22 may include a biosensor or the like worn by the driver.

The information acquiring unit 11 constantly acquires, from the detection device 22, information detected by the detection device 22. The information acquiring unit 11 outputs the information acquired from the detection device 22 to the near accident determining unit 12.

The near accident determining unit 12 determines whether or not a near accident has occurred at the vehicle 20 on the basis of the information acquired by the information acquiring unit 11 from the detection device 22. For this determination, the near accident determining unit 12 uses information that makes it possible to determine that the vehicle 20 has exhibited an action that leads to an accident, such as sudden deceleration, sudden acceleration, sudden steering of the vehicle 20, and sudden approach of the vehicle 20 to an object outside the vehicle. The near accident determining unit 12 determines that a near accident has occurred, for example, when there is a predetermined amount of change in the above information within a predetermined period of time. As described above, the criterion for determining whether or not a near accident has occurred is given to the near accident determining unit 12 in advance. The near accident determining unit 12 outputs the determination result of the near accident to the near accident cause classifying unit 13. In addition, in a case where it is determined that a near accident has occurred, the near accident determining unit 12 stores the information regarding the determined near accident in the storage unit 16. The information regarding the determined near accident is information detected by the detection device 22 including the sensor, the cameras, and the in-vehicle devices within a certain period of time before, after, and including a time point when the near accident has occurred.

The near accident cause classifying unit 13 determines and classifies the cause of the near accident that has occurred at the vehicle 20 on the basis of the information detected by the detection device 22 that is stored in the storage unit 16. Let us assume that there are three categories: human causes, environmental causes, and unexpected causes. The near accident cause classifying unit 13 outputs the category into which the cause of the near accident is classified to the near accident level determining unit 14.

Human causes refer to those in which a near accident has occurred mainly due to the driver's state, such as the driver's dozing and inattentiveness. Note that the driver's state is detected by the driver monitoring device, the cameras, the biological sensor, or the like included in the detection device 22.

Environmental causes refer to those in which a near accident has occurred due to a matter that can be predicted by the driver, such as road conditions such as points with poor visibility, narrow roads, traffic jams, and construction, the weather, and the degree of congestion of bicycles and pedestrians around the vehicle 20. Note that road conditions such as points with poor visibility, narrow roads, traffic jams, and construction are detected by a navigation device included in the detection device 22. The weather is acquired from a server device that provides weather data, for example, by the in-vehicle device included in the detection device 22. The degree of congestion of bicycles and pedestrians around the vehicle 20 is detected by the camera or the like included in the detection device 22.

Unexpected causes refer to those in which a near accident has occurred due to a matter that cannot be predicted by the driver, such as abruptly appearing pedestrians and falling objects. Abruptly appearing pedestrians and falling objects are detected by a corner sensor or the like included in the detection device 22.

For example, in a case where the near accident determining unit 12 determines that a near accident has occurred due to sudden deceleration, it is estimated that the driver has been drowsy and driving while dozing if the driver's eye-opening degree has been small in a certain period of time preceding the occurrence of the near accident. In this case, the near accident cause classifying unit 13 determines that the near accident has occurred due to the driver's dozing and classifies the near accident as a human cause. In addition, if the area around the vehicle 20 has been congested when the near accident has occurred, the near accident cause classifying unit 13 determines that the traffic jam is also the cause and classifies the near accident as an environmental cause in addition to the human cause. In this manner, the near accident cause classifying unit 13 may classify a near accident into a plurality of causes.

Furthermore, for example, in a case where the near accident determining unit 12 determines that a near accident has occurred due to sudden deceleration at a point with poor visibility, the near accident cause classifying unit 13 determines that the driver could have predicted the near accident and classifies the near accident as an environmental cause. In addition, in a case where it is determined that a bicycle or the like has abruptly appeared on the basis of the surrounding situation immediately before the occurrence of this near accident, the near accident cause classifying unit 13 classifies this near accident as an unexpected cause in addition to the environmental cause.

The near accident level determining unit 14 determines the level of the near accident that has occurred at the vehicle 20 on the basis of the information detected by the detection device 22 that is stored in the storage unit 16. The level of a near accident is an index that indicates the priority for a notification regarding the near accident, and the higher the level, the higher the priority.

In addition, the near accident level determining unit 14 determines, for each of combinations of the cause of the near accident that has occurred at the vehicle 20 and a target individual to be notified, whether or not the level of the near accident exceeds a threshold level that is predetermined for the corresponding combination. The details of the threshold level will be described later. The near accident level determining unit 14 outputs the determination result to the notification destination determining unit 15.

In a case where the level of the near accident that has occurred at the vehicle 20 exceeds the threshold level, the notification destination determining unit 15 determines a target individual to be notified of information regarding the near accident (hereinafter referred to as “near accident information”). Note that the target individual is the driver of the vehicle 20 in the example of FIG. 1. The notification destination determining unit 15 may further determine the timing of notifying the target individual of the near accident information. The notification destination determining unit 15 generates near accident information on the basis of the information detected by the detection device 22 that is stored in the storage unit 16 and outputs the generated near accident information to the notification unit 21 at the determined timing. Specific examples of near accident information will be described later.

The storage unit 16 stores information regarding the near accident determined by the near accident determining unit 12. This information is used for, for example, generation of near accident information and calculation of the skill or driving tendency of a driver. The storage unit 16 may further store information regarding the driver's skill and other information. The storage unit 16 may further store the category into which the cause of the near accident is classified by the near accident cause classifying unit 13 and the level of the near accident determined by the near accident level determining unit 14.

The notification unit 21 is a display device in a navigation device or the like mounted on the vehicle 20 or a display device in a mobile terminal such as a smartphone carried by the driver or other passengers of the vehicle 20. The notification unit 21 notifies the target individual to be notified by displaying the near accident information received from the notification destination determining unit 15 on a screen. Alternatively, the notification unit 21 may be a speaker that outputs warning sound, for example.

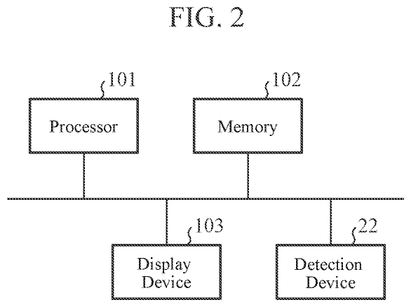

FIG. 2 is a diagram illustrating an exemplary hardware configuration of the notification device 1 according to the first embodiment. The storage unit 16 in the notification device 1 corresponds to a memory 102, and the notification unit 21 corresponds to a display device 103. Meanwhile, the functions of the information acquiring unit 11, the near accident determining unit 12, the near accident cause classifying unit 13, the near accident level determining unit 14, and the notification destination determining unit 15 in the notification device 1 are implemented by a processor 101 that executes a program stored in the memory 102.

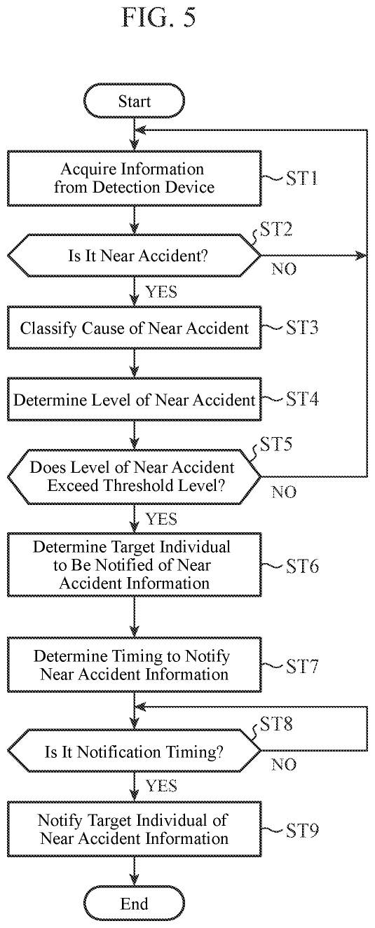

The functions of the information acquiring unit 11, the near accident determining unit 12, the near accident cause classifying unit 13, the near accident level determining unit 14, and the notification destination determining unit 15 are implemented by software, firmware, or a combination of software and firmware. The software or the firmware is described as a program, which is stored in the memory 102. The processor 101 reads and executes a program stored in the memory 102 and thereby implements the functions of the above units. That is to say, the notification device 1 includes the memory 102 for storing a program, execution of which by the processor 101 results in execution of the steps illustrated in the flowchart of FIG. 5 which will be described later. Furthermore, it can be said that this program causes a computer to execute the procedures or the methods of the information acquiring unit 11, the near accident determining unit 12, the near accident cause classifying unit 13, the near accident level determining unit 14, and the notification destination determining unit 15.

In this embodiment, the processor 101 refers to, for example, a central processing unit (CPU), a processing device, an arithmetic device, or a microprocessor.

The memory 102 may be a nonvolatile or volatile semiconductor memory such as a random access memory (RAM), a read only memory (ROM), an erasable programmable ROM (EPROM), or a flash memory, a magnetic disk such as a hard disk or a flexible disk, or an optical disk such as a compact disc (CD) or a digital versatile disc (DVD).

Next, a scene example will be described in which the notification device 1 according to the first embodiment is utilized.

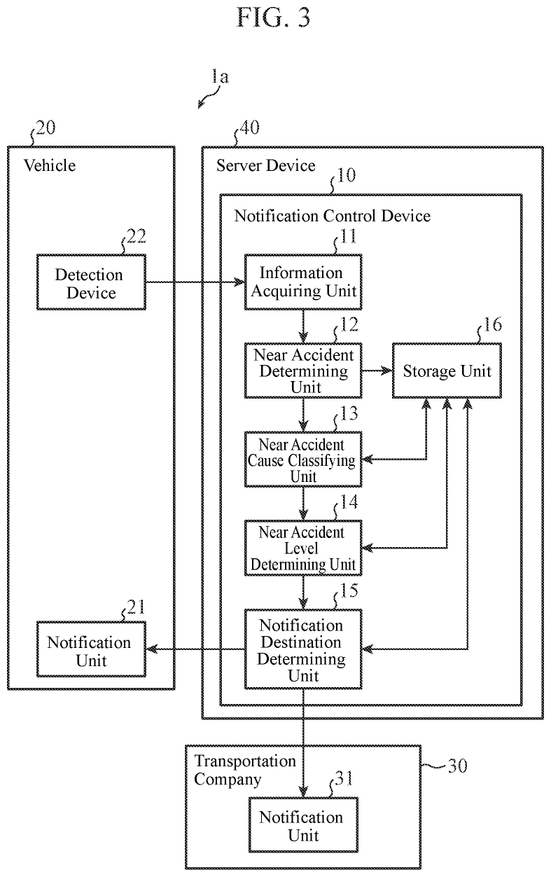

FIG. 3 is a block diagram illustrating an exemplary configuration of a notification system 1a according to the first embodiment. The notification system la is used for fleet management (vehicle management) of a transportation company 30, for example. A manager of the transportation company 30 manages drivers of one or more vehicles 20 using the notification system 1a. In the example of FIG. 3, a notification control device 10 is configured in a server device 40, and the notification control device 10 communicates with a notification unit 21 and a detection device 22 of the vehicle 20 and a notification unit 31 of the transportation company 30 wirelessly or by wire. The notification control device 10 includes an information acquiring unit 11, a near accident determining unit 12, a near accident cause classifying unit 13, a near accident level determining unit 14, a notification destination determining unit 15, and a storage unit 16. The information acquiring unit 11 acquires information from the detection device 22 mounted on each of the vehicles 20. The notification destination determining unit 15 can output the near accident information to the notification unit 21 mounted on each vehicle 20 and the notification unit 31 of the transportation company 30. Note that the notification unit 31 is, for example, a display device of a computer that can be operated by the manager of the transportation company 30 or a display device of a mobile terminal carried by the manager.

In the notification system 1a utilized for fleet management, for example, in a case where a near accident occurs at a certain vehicle 20, the notification control device 10 notifies the manager of the near accident information through the notification unit 31. The manager can check the notified near accident information and give guidance to the driver of the vehicle 20. The notification control device 10 can also warn the driver of the fact that the near accident has occurred through the notification unit 21 of the vehicle 20 and thereby help improve the driver's awareness. Furthermore, since the information regarding the near accident that has occurred at the vehicle 20 is stored in the storage unit 16 of the notification control device 10, the information can be viewed by both the manager and the driver. The information that can be viewed includes, for example, the point where the near accident has occurred and the situation where the near accident has occurred.

It is merit of the notification system 1a according to the first embodiment that the notification destination of the near accident that has occurred at the vehicle 20 that is traveling can be changed depending on various conditions in such fleet management. In this notification system 1a, target individuals to be notified of the near accident include the manager of the transportation company 30 and a driver of each of the vehicles 20.

Note that merit of the notification device 1 of FIG. 1, in which the driver is not managed by a manager, is that whether or not to notify near accident information can be changed depending on, for example, the driving tendency of the driver of the vehicle 20. In this notification device 1, the target individual to be notified of a near accident is the driver of the vehicle 20.

Here, the threshold level for a near accident will be described by taking, as an example, a case where target individuals to be notified of the near accident include the manager of the transportation company 30 and a driver of each of the vehicles 20.

As described above, the near accident level determining unit 14 determines the level of the near accident that has occurred and determines whether or not the level exceeds a predetermined threshold level. At this point, the near accident level determining unit 14 determines the level of the near accident that has occurred by using a plurality of pieces of information in combination, the pieces of information being causes of the near accident that has occurred. A near accident of a low level has a low priority for notification, and the higher the level, the higher the priority for notification.

For example, in a case of a near accident caused by dozing, it is assumed that the information used for classification of the cause of the near accident includes information on the driver's eye-opening degree, sudden braking information, and sudden steering information. In this manner, the information used for classification of the cause of the near accident is predetermined for each cause. In the above case, if the situation where the eye-opening degree is lower than or equal to a certain value lasts for a certain period of time or more at the time immediately before the occurrence of the near accident, the near accident is classified into one of levels of ten stages, for example, depending on the eye-opening degree and time duration, for example. For example, in a case where the driver has closed his eyes for three seconds several times, the near accident cause classifying unit 13 determines that the driver has been dozing, and the near accident level determining unit 14 determines that the degree of dozing is level “6”. In this manner, it is assumed that the levels are predetermined depending on, for example, the number of seconds the eyes have been closed and the number of times the eyes have been closed.

Similarly, also for each of sudden braking and sudden steering, the near accident is classified into one of levels of ten stages, for example, depending on the amount of a change in value at the moment when the sudden braking or the sudden steering has occurred or immediately before the occurrence. For example, a case where the speed changes by 10 km/h in two seconds is regarded as level “3”, and a case where the speed changes by 30 km/h in two seconds is regarded as level “7”. Meanwhile, the near accident may be classified into one of levels depending on the speed when the near accident has occurred and the speed limit of the road. For example, a case of traveling at 40 km/h on a road with a speed limit of 70 km/h is regarded as level “3”, whereas a case of traveling at 70 km/h on the road is regarded as level “7”. Also, for example, a case of traveling at 40 km/h on a road with a speed limit of 70 km/h is regarded as level “3”, whereas a case of traveling at 40 km/h on a road with a speed limit of 40 km/h is regarded as level “7”.

Furthermore, in a case where there is a plurality of causes of a near accident, the near accident level determining unit 14 determines the final level by combining the levels of the respective causes. In the above example, in a case where dozing is level “6”, sudden braking is level “8”, and sudden steering is level “4”, the near accident level determining unit 14 combines these levels and thereby determines that the final level is “7”. As a method of combining, for example, a matrix table or the like is used in which a level is defined for each combination of the cause of a near accident and a value such as the eye-opening degree. It is assumed that the matrix table or the like is given to the near accident level determining unit 14 in advance.

Next, the near accident level determining unit 14 compares the level of the near accident determined as described above with a predetermined threshold level. It is assumed that this threshold level is predetermined for each combination of the cause of a near accident and a target individual to be notified. That is to say, a matrix table or the like, in which a threshold level is defined for each combination of the cause of a near accident and a target individual to be notified, is given to the near accident level determining unit 14 in advance.

First, the relationship between the cause of a near accident and a threshold level will be explained. Here, for the explanation, a near accident caused by inattentiveness is used as an example of a near accident of a human cause, and a near accident caused by a falling object is used as an example of a near accident of an unexpected cause. As the level of a near accident caused by inattentiveness, for example, ten stages are defined which correspond to combinations of, for example, the numerical value of the degree of inattentiveness, the current speed of the vehicle 20, and the behavior of the vehicle 20 at the time when the near accident has occurred. It is assumed that the same applies to the level of a near accident caused by a falling object. Furthermore, a threshold level is also predetermined which indicates the minimum of the level of the near accident that has occurred required to notify a target individual. The threshold level differs for each cause of the near accident, and thus the threshold level differs between a near accident of the human cause and a near accident of the unexpected cause. For example, when a near accident caused by inattentiveness occurs, a target individual is notified when the level of the near accident is “4” or higher, and when a near accident caused by a falling object occurs, a target individual is notified when the level of the near accident is “7” or higher. More specifically, the threshold level is set low for near accidents of the human cause since it is expected that a target individual prefers to receive more notifications for such near accidents. The threshold level is set high for near accidents that have occurred when the traveling speed is high since it is expected that a target individual prefers to receive notifications of serious incidents only which may have led to an accident. Furthermore, a target individual may be able to change the threshold level set in the near accident level determining unit 14.

Next, the relationship between the target individual to be notified and the threshold level will be described.

In a case where the target individual is a driver, it is preferable that the threshold level differs for each driver. This is because it is considered that the likelihood to cause a near accident differs between an experienced driver and an underexperienced driver or between a driver who frequently dozes and a driver who does not, depending on the skill or the driving tendency. It is conceivable that a good driver does not need to be notified of a minor near accident, whereas a driver who is not good needs to be notified of even a minor near accident. It is also conceivable that the manager wants to be notified of even a minor near accident since it is desirable to check and to give guidance to a driver who frequently dozes, from an early stage. Therefore, it is assumed that the near accident level determining unit 14 is provided in advance with threshold levels classified into, for example, ten stages depending on, for example, the skill or the driving tendency of the driver; the near accident level determining unit 14 determines the skill, the driving tendency, or the like of the driver and selects a threshold level depending on the determined skill, the driving tendency, or the like. The near accident level determining unit 14 may calculate the skill of the driver on the basis of, for example, the speed and a deviation of the steering angle among the information which has been detected by the detection device 22 at the time when a near accident has been caused in the past that is stored in the storage unit 16. Alternatively, the manager or the driver may input the skill and thereby store it in the storage unit 16. The near accident level determining unit 14 also determines the driving tendency or the like of the driver on the basis of the information detected by the detection device 22 at the time when a near accident has been caused in the past that is stored in the storage unit 16. Note that the driver or the manager may be allowed to change the threshold level set in the near accident level determining unit 14.

In a case where the target individual is a manager, it is preferable that the threshold level is different for each manager. For example, the threshold level differs depending on the position of a manager so that a low threshold level is set for a section chief who normally manages drivers to notify even a minor near accident and a high threshold level is set for a department manager who manages only serious cases to notify only serious near accidents. Moreover, the manager may be allowed to change the threshold level set in the near accident level determining unit 14.

In a case where the near accident level determining unit 14 determines that the level of a near accident exceeds the threshold level, the notification destination determining unit 15 determines a target individual to be notified of the near accident information. The notification destination determining unit 15 determines the target individual to be notified depending on, for example, the level of the near accident. More specifically, a manager is notified when the level exceeds “5”, whereas a driver is notified when the level exceeds “7”. In this manner, the notification level is predetermined for each target individual. That is to say, in a case where the level of a near accident that has occurred is “7”, the target individuals to be notified of the near accident information are both the manager and the driver, and in a case where the level is “6”, the target individual to be notified is the manager only. Note that the target individual may be allowed to change the notification level for each target individual.

In addition, the notification destination determining unit 15 may determine a target individual to be notified depending on, for example, the cause of a near accident. More specifically, the notification destination determining unit 15 determines that a near accident of a human cause is notified only to the manager and not to the driver. This is a situation in which the notification system 1a does not need to notify the driver since the notification system 1a notifies the manager when the near accident occurs and the manager gives direct guidance to the driver by telephone or by other means. Meanwhile, for example, the notification destination determining unit 15 determines that a near accident of an environmental cause is notified only to the manager and not to the driver. This is a situation in which notification of near accident information of a human cause is unnecessary for the manager since the manager prefers to check only near accident information caused by the actual road conditions and to exclude the near accident information of the human cause. Furthermore, for example, the notification destination determining unit 15 determines that a near accident of an unexpected cause is notified to both the manager and the driver. This is a situation in which the manager prefers to extract a near accident that has occurred in a different situation from usual and to give guidance to the driver, and the driver also prefers to extract and check the near accident that has occurred in a different situation from usual. Note that the target individual may be allowed to change the notification destination for each cause. In addition, the notification destination determining unit 15 may use a matrix table or the like in which a target individual to be notified is defined for each combination of the level of a near accident and the cause of the near accident.

Subsequently, the notification destination determining unit 15 generates near accident information using the information stored in the storage unit 16. The near accident information includes information indicating that the near accident has occurred and information indicating the situation in which the near accident has occurred (for example, the cause and the point of occurrence of the near accident). The notification destination determining unit 15 selects notification methods depending on devices that constitute the notification units 21 and 31. The notification method includes an e-mail, pop-up display, and warning sound.

Then, the notification destination determining unit 15 determines notification timing of the near accident information. Here, it is assumed that the notification timing has two options of “immediately when a near accident occurs” and “after a certain period of time”. The option of after a certain period of time is selected in a case where the level of the near accident exceeds the threshold level, but there is no need to notify immediately, and, for example, near accident information for one week is notified at one time every week. This is useful for target individuals who feel discomfort to receive a notification each time a near accident occurs and only need to be notified later at one time.

Here, “notification” will be described. For example, a “notification” and a “warning” are issued to the driver. The “warning” may be included in the “notification”, meanwhile, the “notification” has two types of meaning. The two types of “notification” are notification with a warning meaning to convey that a near accident has occurred and notification with a meaning to convey not only the occurrence of a near accident but also the details of the near accident. Mainly, notification “immediately when a near accident occurs” refers to the former notification, and notification “after a certain period of time” refers to the latter notification.

The notification destination determining unit 15 determines notification timing depending on at least one of the cause of the near accident, the level of the near accident, or the target individual to be notified, which will be described below.

First, an example in which notification timing is determined by the cause of the near accident will be described. The notification destination determining unit 15 determines notification timing of the near accident that has occurred depending on the notification timing defined for each cause of a near accident. For example, in a case where the cause of the near accident that has occurred is a human cause, the notification destination determining unit 15 determines that the notification timing is “immediately when a near accident occurs” since the target individual to be notified needs to be notified immediately. On the other hand, for near accidents of environmental causes and unexpected causes, the notification destination determining unit 15 determines that the notification timing is “after a certain period of time”. Note that the target individual may be allowed to change the notification timing for each cause.

Next, an example of determining notification timing depending on the level of a near accident will be described. In a case where the threshold level for the near accident is “5”, the notification destination determining unit 15 determines that the notification timing is “after a certain period of time” when the level of the near accident that has occurred is “5” or “6”, and determines that the notification timing is “immediately when a near accident occurs” when the level exceeds “7”. This is utilized in a case where, for example, regarding near accidents caused by inattentiveness, a target individual prefers to know near accident information of level equal to “5” or higher, but does not need to know immediately, and thus it is sufficient to notify several pieces of near accident information at one time, whereas near accidents exceeding level “7” are serious and thus are requested to be notified immediately. Note that the target individual may be allowed to change the notification timing for each level of the near accident.

Lastly, an example of determining the notification timing depending on a target individual to be notified will be described. In a case where the target individuals include the manager and the driver, the notification destination determining unit 15 determines that the notification timing for the driver is “immediately when a near accident occurs” when the level of the near accident that has just occurred exceeds the threshold level. This is because, for example, the driver is actually driving and thus physically feels that the near accident has occurred. On the other hand, in a case of operation in which the manager checks the near accident situation of a driver at constant intervals and gives guidance to the driver, the notification destination determining unit 15 determines that the notification timing for the manager is “after a certain period of time”.

In addition, in a case where the target individual is a driver, the notification destination determining unit 15 determines that the notification timing for an underexperienced driver is “immediately when a near accident occurs” and the notification timing for an experienced driver is “after a certain period of time”. This is because, for example, underexperienced drivers are more likely to cause a near accident as compared to experienced drivers.

Note that the target individual may be allowed to change the notification timing for each target individual to be notified.

When the near accident information is input from the notification destination determining unit 15 to the notification unit 21 or the notification unit 31, the notification unit 21 or the notification unit 31 displays the near accident information or outputs the near accident information by speech. As described above, the notification unit 21 is, for example, a display device in a navigation device or the like mounted on the vehicle 20, a display device in a mobile terminal such as a smartphone carried by the driver or other passengers of the vehicle 20, or a speaker. The notification unit 31 is, for example, a display device of a computer that can be operated by the manager, a display device of a mobile terminal such as a smartphone carried by the manager, or a speaker.

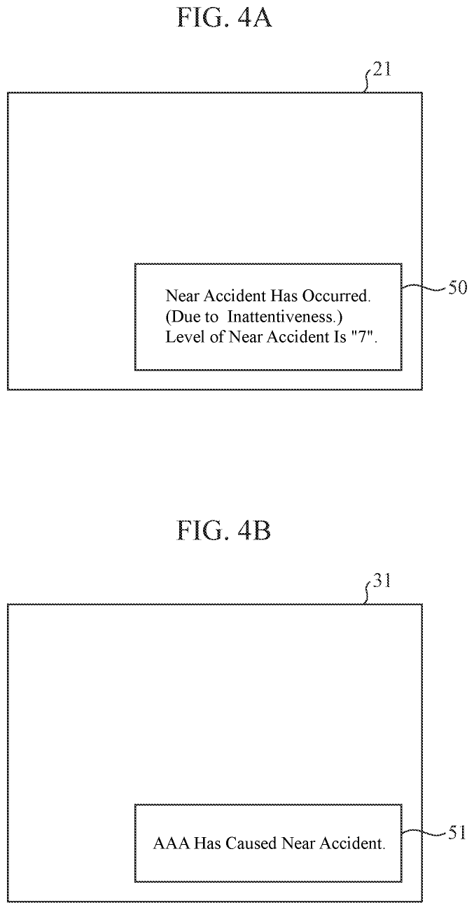

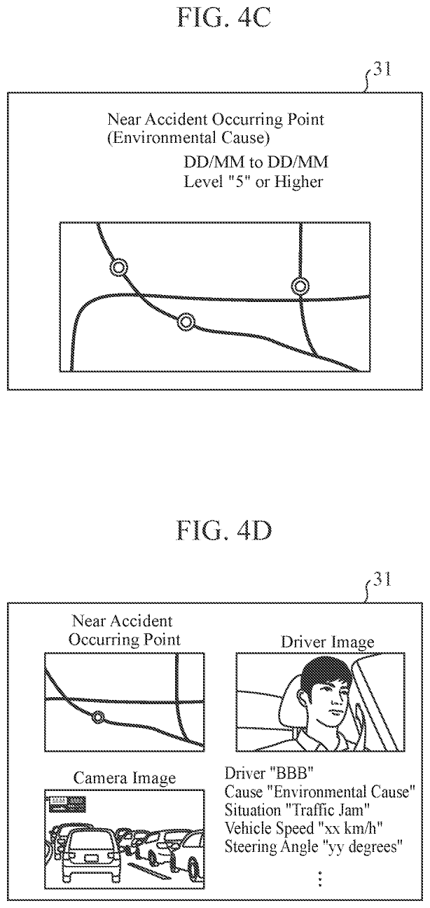

FIG. 4A is a diagram illustrating an example of near accident information displayed by the notification unit 21 in the first embodiment. In the example of FIG. 4A, as soon as a near accident exceeding a threshold level occurs at the vehicle 20, a pop-up image 50 of near accident information is superimposed and displayed on a display screen of a navigation device which is the notification unit 21.

FIG. 4B is a diagram illustrating an example of near accident information displayed by the notification unit 31 in the first embodiment. In the example of FIG. 4B, as soon as a near accident exceeding a threshold level occurs at the vehicle 20, a pop-up image 51 of near accident information is superimposed and displayed on a display screen of a computer of the manager which is the notification unit 31. The letter string of “AAA” in the pop-up image 51 is the name of the driver of the vehicle 20 managed by the manager.

FIG. 4C is a diagram illustrating another example of near accident information displayed by the notification unit 31 in the first embodiment. In the example of FIG. 4C, information regarding near accidents of levels exceeding threshold levels that have occurred at vehicles 20 subjected to management within a certain period of time is displayed on the display screen of the manager's computer which is the notification unit 31. In this screen example, points where near accidents classified as the environmental cause occurred are indicated by double circles (⊚) on a map. Furthermore, for example, in a case where the manager performs operation of selecting one of the near accident occurring points in the screen example of FIG. 4C, the notification unit 31 may display a screen as illustrated in FIG. 4D which indicates the details of the selected near accident occurring point. Displayed in the example of FIG. 4D are a map of the near accident occurring point selected by the manager, an image photographed at the time of occurrence of the near accident by the camera of the vehicle 20 at which the near accident occurred, an image of the driver's face orientation, the name of the driver of the vehicle 20, the cause of the near accident, the situation, etc. Note that the screen examples of FIGS. 4C and 4D may be displayed on a display screen of the navigation device or the like which is the notification unit 21. In addition, selecting the pop-up image 50 of FIG. 4A or the pop-up image 51 of FIG. 4B may result in a switch to the screen display as illustrated in FIG. 4D.

Next, the operation of the notification system 1a will be described.

FIG. 5 is a flowchart illustrating an operation example of the notification system 1a according to the first embodiment. The notification system 1a repeats the operation illustrated in the flowchart of FIG. 5.

In step ST1, the information acquiring unit 11 acquires information from the detection device 22.

In step ST2, the near accident determining unit 12 determines whether or not a near accident has occurred using the information acquired in step ST1. If the near accident determining unit 12 determines that a near accident has occurred (“YES” in step ST2), the process proceeds to step ST3, and if it is determined that no near accident has occurred (“NO” in step ST2), the process returns to step ST1.

In step ST3, the near accident cause classifying unit 13 classifies the cause of the near accident that has been determined in step ST2.

In step ST4, the near accident level determining unit 14 determines the level of the near accident that has been determined in step ST2.

In step ST5, the near accident level determining unit 14 determines whether or not the level of the near accident exceeds a corresponding one of threshold levels. If the near accident level determining unit 14 determines that the level of the near accident exceeds the threshold level (“YES” in step ST5), the process proceeds to step ST6, and if it is determined that the level of the near accident exceeds none of corresponding threshold levels (“NO” in step ST5), the process returns to step ST1.

In step ST6, the notification destination determining unit 15 determines a target individual to be notified of near accident information of the near accident that has been determined to exceed the threshold level in step ST5.

In step ST7, the notification destination determining unit 15 determines the timing of notifying the near accident information. Then, if it is timing to notify the near accident information (“YES” in step ST8), the notification destination determining unit 15 outputs the near accident information to the notification unit 21 or the notification unit 31 corresponding to the target individual to be notified in step ST9. The notification unit 21 or the notification unit 31 notifies the target individual of the near accident information from the notification destination determining unit 15. On the other hand, if it is not the timing to notify the near accident information (“NO” in step ST8), the notification destination determining unit 15 waits until the notification timing is reached.

As described above, the notification control device 10 according to the first embodiment includes the information acquiring unit 11, the near accident determining unit 12, the near accident cause classifying unit 13, the near accident level determining unit 14, and the notification destination determining unit 15. The information acquiring unit 11 acquires information from the detection device 22 mounted on the vehicle 20. The near accident determining unit 12 determines whether or not a near accident has occurred at the vehicle 20 by using the information acquired by the information acquiring unit 11. The near accident cause classifying unit 13 classifies the cause of the near accident determined by the near accident determining unit 12 into a corresponding one of causes. The near accident level determining unit 14 determines the level of the near accident determined by the near accident determining unit 12 and also determines whether or not the level of the near accident exceeds a corresponding one of predetermined threshold levels. The notification destination determining unit 15 determines one of target individuals who is to be notified of information regarding the near accident that is determined to exceed the threshold level by the near accident level determining unit 14. In this configuration, since the threshold levels are set for respective combinations each of which is a combination of a corresponding one of the causes and a corresponding one of the target individuals, the notification control device 10 can eliminate unnecessary notifications for target individuals and can also allow the notification units 21 and 31 to perform appropriate notification with distinction among target individuals.

The notification system 1a according to the first embodiment includes the notification control device 10 and the notification units 21 and 31 for notifying a target individual determined by the notification control device 10 of near accident information. Meanwhile, the notification device 1 according to the first embodiment includes the notification control device 10 and the notification unit 21 for notifying a target individual determined by the notification control device 10 of near accident information. With these configurations, the notification system 1a and the notification device 1 can perform appropriate notification with distinction among target individuals.

Moreover, the notification destination determining unit 15 of the first embodiment determines a target individual to be notified of the near accident information depending on the cause into which the near accident is classified by the near accident cause classifying unit 13. As a result, the notification system 1a and the notification device 1 can eliminate unnecessary notifications for the target individual.

Furthermore, the notification destination determining unit 15 of the first embodiment determines timing to notify the target individual depending on at least one of the cause into which the near accident is classified by the near accident cause classifying unit 13, the level determined by the near accident level determining unit 14, or the target individual determined by the notification destination determining unit 15 itself. As a result, the notification system 1a and the notification device 1 can perform notification at appropriate timing.

In addition, target individuals in the first embodiment include the driver of the vehicle 20 and the manager who manages the driving of the vehicle 20. As a result, the notification system 1a can be utilized for fleet management.

Note that the present invention may include a modification of any component of the embodiment or omission of any component of the embodiment within the scope of the invention.

For example, the server device 40 includes the notification control device 10 in the example of FIG. 3; however, each of the vehicles 20 may include the notification control device 10, or the transportation company 30 may include the notification control device 10. Alternatively, the information acquiring unit 11, the near accident determining unit 12, the near accident cause classifying unit 13, the near accident level determining unit 14, the notification destination determining unit 15, and the storage unit 16 of the notification control device 10 may be distributed among the vehicle 20, the transportation company 30, and the server device 40.

### INDUSTRIAL APPLICABILITY

A notification device according to the present invention is suitable for a notification device used for, for example, fleet management since appropriate notification is performed with distinction among target individuals.

### REFERENCE SIGNS LIST

1: notification device, 1a: notification system, 10: notification control device, 11: information acquiring unit, 12: near accident determining unit, 13: near accident cause classifying unit, 14: near accident level determining unit, 15: notification destination determining unit, 16: storage unit, 20: vehicle, 21: notification unit, 22: detection device, 30: transportation company, 31: notification unit, 40: server device, 101: processor, 102: memory, 103: display device

1. A notification control device comprising:
a processor to execute a program; and
a memory to store the program which, when executed by the processor, performs processes of,
acquiring information from a detection device mounted on a vehicle;
determining whether or not a near accident has occurred at the vehicle using the information acquired;
classifying a cause of the near accident determined into a corresponding one of causes;
determining a level of the near accident determined and determining whether or not the level of the near accident exceeds a corresponding one of predetermined threshold levels; and
determining one of target individuals who is to be notified of information regarding the near accident that is determined to exceed the corresponding one of the predetermined threshold levels,
wherein the predetermined threshold levels are set for respective combinations each of which is a combination of a corresponding one of the causes and a corresponding one of the target individuals.

2. The notification control device according to claim 1, wherein the processes further include classifying the near accident into at least one of a human cause that is attributable to a driver of the vehicle, an environmental cause that is attributable to a matter from which the driver has been able to predict occurrence of the near accident, or an unexpected cause that is attributable to a matter from which the driver has not been able to predict occurrence of the near accident.

3. The notification control device according to claim 1, wherein the processes further include determining the one of the target individuals who is to be notified of the information regarding the near accident depending on the one of the causes into which the cause of the near accident is classified.

4. The notification control device according to claim 1, wherein the processes further include determining timing to notify the determined one of the target individuals depending on at least one of the one of the causes into which the cause of the near accident is classified, the level determined, or the determined one of the target individuals.

5. The notification control device according to claim 1, wherein the target individuals include a driver of the vehicle and a manager who manages driving of the vehicle.

6. A notification device comprising:
the notification control device according to claim 1; and
a display or a speaker to notify the information regarding the near accident to the one of the target individuals who is determined by the notification control device.

7. The notification device according to claim 6, wherein the display to display the information regarding the near accident is included.

8. A notification system comprising:
a processor to execute a program; and
a memory to store the program which, when executed by the processor, performs processes of,
acquiring information from a detection device mounted on a vehicle;
determining whether or not a near accident has occurred at the vehicle using the information acquired;
classifying a cause of the near accident determined into a corresponding one of causes;
determining a level of the near accident determined and determining whether or not the level of the near accident exceeds a corresponding one of predetermined threshold levels;
determining one of target individuals who is to be notified of information regarding the near accident that is determined to exceed the corresponding one of the predetermined threshold levels; and
a display or a speaker to notify the information regarding the near accident to the one of the target individuals who is determined,
wherein the predetermined threshold levels are set for respective combinations each of which is a combination of a corresponding one of the causes and a corresponding one of the target individuals.

9. A notification control method comprising:
acquiring information from a detection device mounted on a vehicle;
determining whether or not a near accident has occurred at the vehicle using the information acquired;
classifying a cause of the near accident determined into a corresponding one of causes;
determining a level of the near accident determined and determining whether or not the level of the near accident exceeds a corresponding one of predetermined threshold levels; and
determining one of target individuals who is to be notified of information regarding the near accident that is determined to exceed the corresponding one of the predetermined threshold levels,
wherein the predetermined threshold levels are set for respective combinations each of which is a combination of a corresponding one of the causes and a corresponding one of the target individuals.
